(19) United States
(12) Patent Application Publication
(10) Pub. No.: US 2015/0005793 A1
(43) Pub. Date: Jan. 1, 2015

(54) Devices and Methods for Removing Cerumen from an Ear

(71) Applicants: Andrew D. Collins, Brentwood, MO (US); John Edward Collins, Lafayette, LA (US)

(72) Inventors: Andrew D. Collins, Brentwood, MO (US); John Edward Collins, Lafayette, LA (US)

(21) Appl. No.: 13/928,101

(22) Filed: Jun. 26, 2013

(51) Int. Cl. A61F 11/00

(52) U.S. Cl. CPC A61F 11/006
USPC 606/162

(57) ABSTRACT

A device sized to be handled by a user for cleaning wax from an ear. The device includes a central handle that facilitates handling by the user. A first wire loop extends outward from the first end of the handle, and a second wire loop extends outward from the second end of the handle. The wire loops are deformable to allow the user to tailor the shape and size for insertion into the ear and removal of wax.

### BACKGROUND

The present application is directed to devices and methods for cleaning cerumen from a patient's ear and, more particularly, to a device with at least one deformable loop.

Medical practitioners needing to examine the ear canal and tympanic membrane of patients often use curettes to remove cerumen from the ear canal. A clear view of the tympanic membrane is necessary in order for a practitioner to determine if a patient has an infection of the middle ear, a perforation of the tympanic membrane, or other diseases or injuries of the ear. Cerumen removal is one of the most common procedures done by pediatricians, family practitioners, and other primary care providers.

Various devices have been used in the past to remove cerumen from the ear. These devices include an elongated shaft with an enclosed eyelet at one end. The shaft provides for inserting the eyelet into the patient's ear where the cerumen can be removed in the eyelet. However, these designs include a variety of drawbacks.

One issue with the current designs is the inability to change the shape of the device depending upon the specific needs of the patient. The devices include a fixed shape that cannot be changed. This limits the utility of the devices, and also diminishes their effectiveness in removing cerumen.

Further, the current devices do not meet the needs of the medical practitioners. Many practitioners do not find the devices effective, and therefore alter the designs of other devices to meet their needs. Many times these altered devices are more effective, but still do not completely satisfy the needs of the practitioners.

### SUMMARY

The present application is directed to devices and methods for removing cerumen from the ear. One embodiment includes an ear cleaning device that includes a central handle, a continuous first loop that extends outward from a first end of the handle, and a continuous second loop that extends outward from an opposing second end of the handle. Each of the first and second loops includes spaced-apart first and second arms that extend from the handle and connect together at a curved terminal end. The first and second loops are constructed from a common wire, and the handle is constructed from a different material than both of the first and second loops.

The wire may include first and second spaced apart ends that are each positioned within the handle.

The wire may be deformable.

The wire may be a continuous piece with no ends.

The first and second arms of each of the loops may include straight sections that extend outward from the handle with the straight sections being parallel.

The width of the curved terminal end of each of the first and second loops may be larger than a distance between the first and second arms at the handle.

Each of the first and second loops may include identical shapes and sizes.

The wire may overlap at least once between the first and second ends of the handle.

Another embodiment is directed to an ear cleaning device that includes a handle having opposing first and second ends, a first loop that extends outward from a first end of the handle and is constructed from a first wire, and a second loop that extends outward from an opposing second end of the handle and is constructed from a second wire. The second wire includes a different rigidity than the first wire. The handle is constructed from a different material than each of the first and second wires. An inner portion of each of the first and second loops is positioned within an interior of the handle.

The first and second loops may be isolated from one another within the interior of the handle.

The first wire may include a different gauge than the second wire.

Each of the first and second loops may include arms that extend outward from the handle and a terminal curved end.

Each of the first and second wires may include a circular cross-sectional shape.

Another embodiment is directed to a method of cleaning an ear that includes grasping a device that includes a handle, a first loop that extends outward from a first end of the handle, and a second loop that extends outward from a second end of the handle. Each of the first and second loops initially includes a pair of arms that extend outward from the handle and a curved outer end. The method also includes deforming the first loop from an initial first loop shape, deforming the second loop from an initial second loop shape, inserting the deformed first loop into the ear, and inserting the deformed second loop into the ear.

The second loop may be deformed after the first loop has been inserted into the ear.

The first loop may be formed from a first material and the second loop may be formed from a different second material with a different deformation characteristic.

The first and second loops may initially each include the same shape.

The method may include deforming the first loop by twisting the first loop such that the pair of arms overlap at least once between the handle and the curved outer end.

The method may include deforming the first loop into a first deformed shape and deforming the second loop into a different second deformed shape.

The method may include applying a force to the first loop and deforming the first loop without the force deforming the second loop.

These various aspects may be used together or individually.

### BRIEF DESCRIPTION OF THE DRAWINGS

FIG. 1 is a perspective view of a device.

FIG. 1B is a perspective view of the device of FIG. 1 in a deformed orientation.

FIG. 2 is a top schematic sectional view of a device.

FIG. 3 is a top schematic sectional view of a device.

FIG. 4 is a top schematic sectional view of a device.

FIG. 5 is a top schematic sectional view of a device.

FIG. 6A is a top view of a device.

FIG. 6B is a side view of the device of FIG. 6A in a deformed orientation.

FIG. 7A is a top view of a device.

FIG. 7B is a top view of the device of FIG. 7A in a deformed orientation.

FIG. 8 is a top schematic sectional view of a device.

### DETAILED DESCRIPTION

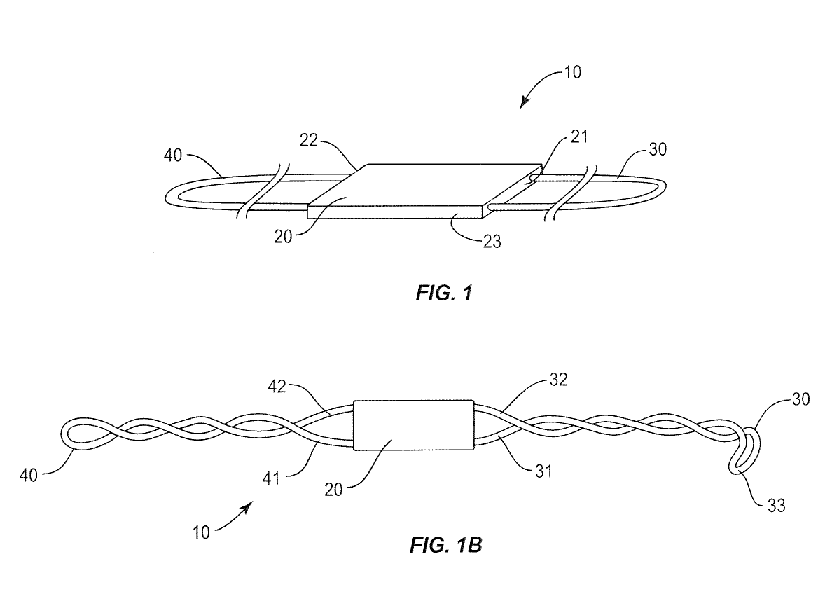

The present application is directed to a device for cleaning wax from an ear. FIG. 1 illustrates a device 10 that is sized to be handled by a user. The device 10 includes a central handle 20 that facilitates handling by the user. A first wire loop 30 extends outward from the first end of the handle 20, and a second wire loop 40 extends outward from the second end of the handle 20. The wire loops 30, 40 are deformable to allow the user to tailor the shape and size for insertion into the ear and removal of wax.

FIG. 1B illustrates the device 10 of FIG. 1 after it has been deformed to accommodate the user. In this embodiment, both the first and second loops 30, 40 have been twisted to change the shape and rigidity to more effectively insert into the ear. The end 33 of the first loop 30 has been further bent for more effective insertion into the ear and removal of ear wax. It is understood that FIG. 1B illustrates one embodiment of the deformation of the device 10. The user is able to deform one or both loops 30, 40 as necessary to accommodate their needs.

The handle 20 is centrally located between the loops 30, 40. The handle 20 is sized and shaped to be grasped and manipulated by the user. The handle 20 includes a first end 21 that faces towards the first loop 30, and a second end 22 that faces towards the second loop 40. The exterior of the handle 20 may include knurled or roughened surfaces to facilitate grasping by the user. The handle 20 may be flat or off-round such that the device 10 will not rotate when being handled by the user. This type of shape may also allow the user to be aware of the orientation of the loops 30, 40 when the user cannot visually see the device 10.

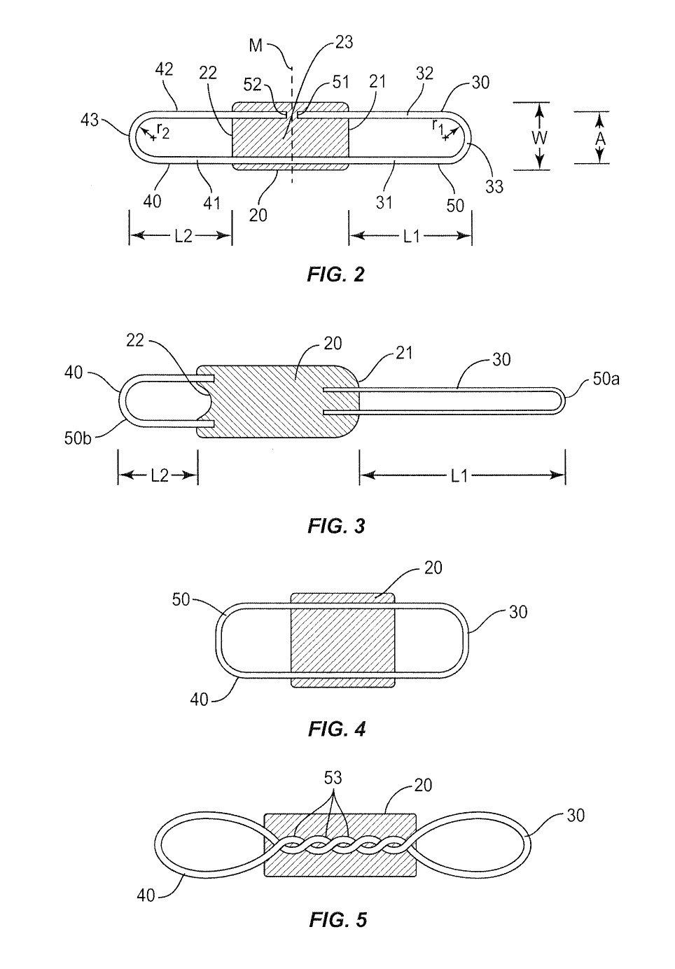

The length, width, and thickness of the handle 20 may vary depending upon the needs of the user. FIG. 2 illustrates an embodiment in which the width W of the handle 20 is greater than the widths A of both the first and second loops 30, 40.

The handle 20 also includes an interior section 23 formed between the outer surfaces. The interior section 23 is sized to receive inner portions of the first and second loops 30, 40. The size of the handle 20 relative to the loops 30, 40 provides for the loops 30, 40 to just extend from the first and second ends 21, 22. The loops 30, 40 are otherwise contained within the handle 20. The handle 20 may be a solid member with a solid interior section 23. Alternatively, the handle 20 may include a hollow interior section 23.

The handle 20 may be made from a variety of materials, including but not limited to plastic, cardboard, metal, and rubber.

The loops 30, 40 extend outward from the opposing ends 21, 22 of the handle 20. As illustrated in FIG. 2, the first loop 30 includes first and second sections 31, 32 and a curved end 33. The curved end 33 includes a radius r1. The first and second sections 31, 32 extend outward from the handle 20. The sections 31, 32 are spaced apart from where they extend from the handle 20 to the terminal end 33. In this embodiment, the sections 31, 32 are substantially straight and parallel with each other. The first loop 31 includes a length L1 measured from the first end 21 to the end 33. The length L1 may vary depending upon the context of use.

The second loop 40 similarly includes first and second sections 41, 42, and a curved end 43. The second loop 40 includes a length L2 and the end 43 includes a curved shape with a radius r2. In this embodiment, the second loop 40 includes the same shape and size as the first loop 30.

The device 20 may be symmetrical about a midline M as illustrated in FIG. 2. This configuration provides for the size and shape of the handle 20 and loops 30, 40 to be the same on each size of the midline M. Other embodiments may include non-symmetrical designs relative to the midline M due to differences in the handle 20 and/or loops 30, 40. FIG. 3 illustrates an embodiment that is non-symmetrical. The handle 20 includes different shapes of the first and second ends 21, 22. Further, the loops 30, 40 include different shapes and sizes. The first loop 30 includes a greater length and narrower width than the second loop 40. Further, the loops 30, 40 include different shapes.

The loops 30, 40 are designed to be deformable to allow the user to alter the shape as necessary to effectively remove wax from the ear. In one embodiment, the loops 30, 40 are constructed from a metal wire. In one embodiment, the wire includes a circular cross-sectional shape. The curved shape provides for smooth surfaces that prevent damaging the ear when the loops 30, 40 are moved across the ear. The gauge of the wire may vary depending upon the needs of the user.

The loops 30, 40 may be made from a variety of materials, including but not limited to metals such as aluminum, plastic, and metal alloys. The material provides for the loops 30, 40 to be deformed by the application of a force. After deformation, the loops 30, 40 maintain the deformed shape. In one embodiment, the loops 30, 40 are constructed from a different material than the handle 20.

The loops 30, 40 may be formed by a single wire. FIG. 2 illustrates an embodiment with a single wire 50 extending from the handle 20 and forming both the first and second loops 30, 40. The wire 50 includes opposing first and second ends 51, 52 that are positioned within the interior 23 of the handle 20. This prevents the ends 51, 52 from being exposed where they could potentially damage the ear during use of the device 10. The ends 51, 52 are spaced apart by a gap. Other embodiments may include the ends 51, 52 being abutted together within the handle interior 23.

FIG. 4 includes another embodiment with the wire 50 being a continuous piece. The continuous piece does not include ends 51, 52. The wire 50 extends from the handle 20 to form the first and second loops 30, 40.

The loops 30, 40 may also be formed by different wires as illustrated in FIG. 3. The first loop 30 is formed by a first wire 50a, and the second loop 40 is formed by a second wire 50b. Each wire includes opposing ends that are positioned within the interior 23 of the handle 20. In embodiments with separate wires, the wires 50a, 50b may include the same or different constructions. One embodiment includes the same type and size of wire being used for each loop 30, 40. Another embodiment includes different types and/or sizes of wire for the different loops 30, 40. In one embodiment, the loops 30, 40 are formed by wires with different deformation characteristics. One of the wires may be more rigid than the other.

In embodiments with a single wire 50 forming each of the loops 30, 40, the wire 50 may include a non-overlapping arrangement within the interior 23 of the handle 20. FIGS. 2 and 4 each disclose non-overlapping arrangements with the sections of the wire 50 remaining spaced apart within the interior 23. Other embodiments as illustrated in FIG. 5 may include an overlapping arrangement in which the wire 50 overlaps at one or more places within the handle 20. The overlap points 53 are positioned within the interior 23 with the two sections 31, 32, 41, 42 being spaced apart at the point where they extend outward from the ends 21, 22 of the handle 20. The overlapping arrangement may provide strength and rigidity to the loops 30, 40.

In use, the user grasps the device 10 at the handle 20. In one embodiment, the user is a medical provider who is working on a patient. In another embodiment, the user is removing wax from their own ears.

The user may initially insert one of the loops 30, 40 in the predefined shape into the ear to determine whether it is able to remove the wax. The user may also deform one or both loops 30, 40 for more effectively removing the wax. The deformation may include changing the shape of the one or both loops 30, 40 in various manners. This may include any one or more of the following: twisting the loops 30, 40 to form overlaps between the handle 20 and the ends 33, 43; squeezing or separating the arms 31, 32, 31,42 relative to each other; and bending one or both ends 33, 43 towards the handle 20. Additional deformations may also occur as necessary by the user.

In one embodiment, the user deforms the first loop for the initial shape to a second shape. The user then inserts the deformed first loop into the ear and removes a first portion of the wax. Afterwards, the first loop is removed from the ear and the user deforms the second loop into a different shape. The user then inserts the deformed second loop into the ear to remove an additional amount of the wax from the ear.

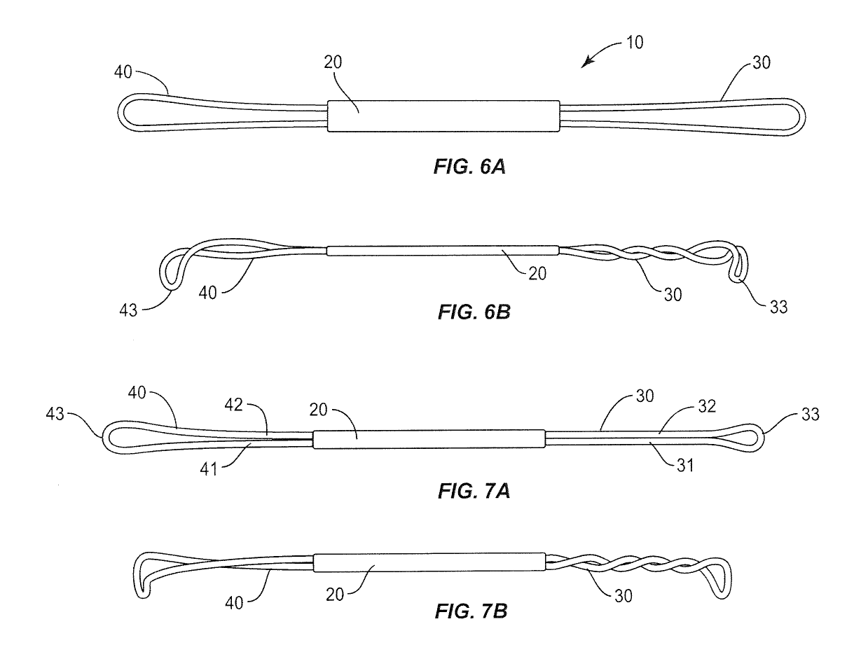

FIG. 6A illustrates one embodiment of a device 10 in its initial shape. The device 10 may be used in this shape for insertion into the ear and removing ear wax. Further, one or both loops 30, 40 may be deformed to better facilitate insertion and removal. FIG. 6B illustrates a side view of the device 10 after deformation. Each of the loops 30, 40 have been twisted. Further, each of the ends 33, 43 have been bent downward as illustrated in FIG. 6B.

FIG. 7A illustrates another version of a device 10. In this initial shape, the first loop 30 includes first and second arms 31, 32 that are in close proximity or in contact. The second loop 40 includes a similar shape with first and second arms 41, 42. Each of the ends 33, 43 includes a rounded shape with an enlarged width. FIG. 7B illustrates the device 10 in a deformed shape. Each loop 30, 40 has been twisted and bent as needed by the user.

Once complete, the device 10 may be discarded by the user. The device 10 is designed to be relatively inexpensive such that a new device 10 is used for every new application.

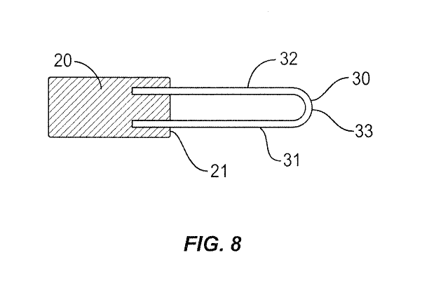

In the embodiments described above, the device 10 includes first and second loops 30, 40 extending from opposing sides of the handle 20. Other embodiments include a single loop 30 extending from the handle 20. The loop 30 extends from the first side of the handle 20. The loop 30 includes first and second arms 31, 32, and a terminal curved end 33. One embodiment of a device 10 with a single loop 30 is illustrated in FIG. 8.

The embodiments described above are used for removing cerumen from the ear. The device 10 may also be used for other applications, including but not limited to procedures involving the mouth and other areas of the body.

The various implants and insertion devices may be used during surgical procedures on living patients. These may also be used in a non-living situation, such as within a cadaver, model, and the like. The non-living situation may be for one or more of testing, training, and demonstration purposes.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. An ear cleaning device comprising:
a central handle;
a continuous first loop that extends outward from a first end of the handle;
a continuous second loop that extends outward from an opposing second end of the handle;
each of the first and second loops including spaced-apart first and second arms that extend from the handle and connect together at a curved terminal end;
the first and second loops being constructed from a common wire;
the handle being constructed from a different material than both of the first and second loops.

2. The ear cleaning device of claim 1, wherein the wire includes first and second spaced apart ends that are each positioned within the handle.

3. The ear cleaning device of claim 1, wherein the wire is deformable.

4. The ear cleaning device of claim 1, wherein the wire is a continuous piece with no ends.

5. The ear cleaning device of claim 1, wherein the first and second arms of each of the loops including straight sections that extend outward from the handle, the straight sections being parallel.

6. The ear cleaning device of claim 1, wherein a width of the curved terminal end of each of the first and second loops is larger than a distance between the first and second arms at the handle.

7. The ear cleaning device of claim 1, wherein each of the first and second loops include identical shapes and sizes.

8. The ear cleaning device of claim 1, wherein the wire overlaps at least once between the first and second ends of the handle.

9. An ear cleaning device comprising:
a handle having opposing first and second ends;
a first loop that extends outward from a first end of the handle, the first loop constructed from a first wire;
a second loop that extends outward from an opposing second end of the handle, the second loop constructed from a second wire;
the second wire including a different rigidity than the first wire;
the handle being constructed from a different material than each of the first and second wires;
an inner portion of each of the first and second loops being positioned within an interior of the handle.

10. The ear cleaning device of claim 9, wherein the first and second loops are isolated from one another within the interior of the handle.

11. The ear cleaning device of claim 9, wherein the first wire includes a different gauge than the second wire.

12. The ear cleaning device of claim 9, wherein each of the first and second loops including arms that extend outward from the handle and a terminal curved end.

13. The ear cleaning device of claim 9, wherein each of the first and second wires includes a circular cross-sectional shape.

14. A method of cleaning an ear comprising:
grasping a device that includes a handle, a first loop that extends outward from a first end of the handle, and a second loop that extends outward from a second end of the handle, each of the first and second loops initially including a pair of arms that extend outward from the handle and a curved outer end;
deforming the first loop from an initial first loop shape;
deforming the second loop from an initial second loop shape;
inserting the deformed first loop into the ear; and
inserting the deformed second loop into the ear.

15. The method of claim 14, wherein the second loop is deformed after the first loop has been inserted into the ear.

16. The method of claim 14, wherein the first loop is formed from a first material and the second loop is formed from a different second material with a different deformation characteristic.

17. The method of claim 14, wherein the first and second loops initially each include the same shape.

18. The method of claim 14, wherein deforming the first loop comprises twisting the first loop such that the pair of arms overlap at least once between the handle and the curved outer end.

19. The method of claim 14, further comprising deforming the first loop into a first deformed shape and deforming the second loop into a different second deformed shape.

20. The method of claim 14, further comprising applying a force to the first loop and deforming the first loop without the force deforming the second loop.
